(19) United States
(12) Patent Application Publication
(10) Pub. No.: US 2023/0180817 A1
(43) Pub. Date: Jun. 15, 2023

(54) A Nicotine Oral Delivery Product

(71) Applicant: JT International SA, Geneva (CH)

(72) Inventor: Maria Ågren, Vårgårda (SE)

(73) Assignee: JT International SA, Geneva (CH)

(21) Appl. No.: 17/921,538

(22) PCT Filed: Apr. 27, 2021
(86) PCT No.: PCT/EP2021/060964

§ 371 Date: Oct. 26, 2022

(30) Foreign Application Priority Data
Apr. 28, 2020 (EP) 20171895.4

(51) Int. Cl. A24B 15/16; A61K 31/465; A61K 9/00; A61K 47/38; A24B 13/00; A24B 15/40

(52) U.S. Cl. CPC A24B 15/16; A61K 31/465; A61K 9/0053; A61K 9/009; A61K 47/38; A24B 13/00; A24B 15/403

(57) ABSTRACT

A nicotine oral delivery product includes a composition enclosed in a water insoluble pouch, wherein the pouch is permeable for saliva and dissolved compounds of the composition, the composition including a source of nicotine and a cellulose or cellulose derivative, wherein the cellulose or cellulose derivative is in granular form. A method of making the nicotine oral delivery product is also provided.

### FIELD OF THE INVENTION

The present invention relates to a nicotine oral delivery product.

### BACKGROUND OF THE INVENTION

Nicotine oral delivery products are known as replacements for smoking articles such as cigarettes. These nicotine oral delivery products provide an alternative, reduced risk, means of administering nicotine to a user in a pleasurable manner.

Nicotine oral delivery products may be provided in loose form, commonly known as snus, or comprise a composition enclosed within a pouch. They are typically placed between the upper or lower gum and lip or cheek of a user, and are retained in this position for a period of time during which, for the pouched nicotine oral delivery products, saliva passes into the interior of the pouch and dissolved components including flavour and nicotine-containing components diffuse therefrom into a user's mouth.

It is desirable to provide a nicotine oral delivery product that effectively delivers nicotine to a user, whilst having an optimal moisture content, a pleasant taste and mouth-feel, and avoiding discolouration of a user's teeth or gums.

### SUMMARY OF THE INVENTION

According to an aspect of the present invention, there is provided a nicotine oral delivery product containing a composition enclosed in a water insoluble pouch, wherein said pouch is permeable for saliva and dissolved components of the composition, the composition comprising a source of nicotine and a cellulose derivative.

According to another aspect of the present invention, there is provided a nicotine oral delivery product containing a composition enclosed in a water insoluble pouch, wherein said pouch is permeable for saliva and dissolved components of the composition, the composition comprising a source of nicotine and a cellulose or cellulose derivative, wherein the cellulose or cellulose derivative is in granular form.

According to a further aspect of the present invention, there is provided a method of making a nicotine oral delivery product containing a composition enclosed in a water insoluble pouch, wherein said pouch is permeable for saliva and dissolved components of the composition, the composition comprising a source of nicotine and a cellulose derivative, and wherein the method comprises enclosing the composition in a water insoluble pouch.

According to a further aspect of the present invention, there is provided a method of making a nicotine oral delivery product containing a composition enclosed in a water insoluble pouch, wherein said pouch is permeable for saliva and dissolved components of the composition, the composition comprising a source of nicotine and a cellulose or cellulose derivative, wherein the cellulose or cellulose derivative is in granular form, and wherein the method comprises introducing the cellulose or cellulose derivative into the composition in granular form, and enclosing the composition in a water insoluble pouch.

According to a still further aspect of the present invention, there is provided a nicotine oral delivery product containing a composition enclosed in a water insoluble pouch, wherein said pouch is permeable for saliva and dissolved components of the composition, the composition comprising a source of nicotine and a cellulose or cellulose derivative, wherein the cellulose or cellulose derivative is introduced into the composition in granular form.

### DETAILED DESCRIPTION OF THE INVENTION

It has been surprisingly and advantageously found that the nicotine oral delivery product of the present invention effectively delivers nicotine to a user, whilst having an optimal moisture content, a pleasant taste and mouth-feel, and avoiding discolouration of a user's teeth or gums.

According to an aspect of the present invention, there is provided a nicotine oral delivery product containing a composition enclosed in a water insoluble pouch, wherein said pouch is permeable for saliva and dissolved components of the composition, the composition comprising a source of nicotine and a cellulose derivative.

According to another aspect of the present invention, there is provided a nicotine oral delivery product containing a composition enclosed in a water insoluble pouch, wherein said pouch is permeable for saliva and dissolved components of the composition, the composition comprising a source of nicotine and a cellulose or cellulose derivative, wherein the cellulose or cellulose derivative is in granular form.

The source of nicotine of the composition of the nicotine oral delivery product of the present invention may be any suitable source of nicotine. The source of nicotine may be a pharmaceutically acceptable source of nicotine. It will be appreciated that the source of nicotine is separate to, i.e. not bound to, the cellulose or cellulose derivative and/or additional cellulose or additional cellulose derivative defined herein. By “source of nicotine” is meant any source providing nicotine in a form that can be solubilised by saliva entering the interior of the pouch such that the nicotine can diffuse therefrom in the saliva and pass into a user's mouth. It is noted that some sources of nicotine may be completely soluble in saliva, whilst others may remain insoluble but provide nicotine in a form that can be solubilised by saliva. The term “soluble in saliva” and like terms used herein simply refers to a component which has a solubility in saliva which is adequate for its intended use, i.e. enables it to be transported from the interior of the pouch and into the oral cavity of a user in saliva.

Examples of suitable sources of nicotine include tobacco, nicotine salt, nicotine base, stabilised nicotine, and combinations thereof. Tobacco includes any part, e.g. leaves, flowers or stems, of any member of the genus Nicotiana, and reconstituted material thereof. Suitable nicotine salts include, but are not limited to the following: nicotine hydrochloride, nicotine dihydrochloride, nicotine monotartrate, nicotine bitartrate, nicotine bitartrate dihydrate, nicotine sulphate, nicotine zinc chloride monohydrate and nicotine salicylate, and mixtures thereof. Nicotine base may be synthetically produced or extracted from tobacco. By “stabilised nicotine” is meant nicotine bound to, adsorbed to, absorbed into, enclosed into or forming a complex or any other non-covalent binding with another component(s). A number of stabilised nicotine combinations are well known in the art. Examples of stabilised nicotine include, but are not limited to: nicotine bound to an ion exchange resin such as a cation exchange resin, for example, Amberlite IRP 64 derived from a copolymer of methacrylic acid and divinylbenzene; nicotine bound to a zeolite; nicotine bound to cellulose and cellulose derivatives, as well as nicotine bound to starch microspheres and beta-cyclodextrin inclusion complexes.

The source of nicotine may be selected from tobacco, nicotine salt, nicotine base and stabilised nicotine, or combinations thereof.

Alternatively, the source of nicotine may be selected from nicotine salt, nicotine base and stabilised nicotine, or combinations thereof, i.e. the source of nicotine, and thus the nicotine oral delivery product of the present invention, is tobacco-free.

Preferably, the source of nicotine of the composition of the nicotine oral delivery product of the present invention is stabilised nicotine. Preferably, the source of nicotine is nicotine polacrilex (nicotine bound to an ion-exchange resin such as Amberlite IRP64 or Purolite C115HMR or Doshion P551).

The source of nicotine may provide from 0.1 to 30 mg of nicotine per nicotine oral delivery product, calculated as nicotine in the form of free base nicotine. Preferably, the source of nicotine provides 1 to 30 mg, such as 2 to 20 mg of nicotine per nicotine oral delivery product, calculated as nicotine in the form of free base nicotine, and more preferably from 3 to 18 mg of nicotine per nicotine oral delivery product, calculated as nicotine in the form of free base nicotine.

The source of nicotine may be present in the composition of the nicotine oral delivery product of the present invention in any suitable amount. Preferably, the composition comprises from 1 to 15 wt % of the source of nicotine, such as from 1 to 10 wt %, or even from 4 to 10 wt % of the source of nicotine.

It has been surprisingly and advantageously found that the effective delivery of nicotine, optimal moisture content, pleasant taste and mouth-feel, and avoidance of discolouration of a user's teeth and gums demonstrated by the nicotine oral delivery product of the present invention may result from the presence of a cellulose derivative in the composition of the nicotine oral delivery product. This is embodied by an aspect of the present invention, but is also applicable to all other aspects of the present invention.

The composition of the nicotine oral delivery product of the present invention may comprise any suitable cellulose derivative. The term “cellulose derivative” as used herein in relation to the composition of the nicotine oral delivery product of the present invention, refers to cellulose which has been modified by virtue of the hydroxyl groups of the compound being partially or fully reacted with one or multiple reagents. Examples of suitable cellulose derivatives include cellulose ethers and cellulose esters. In the production of cellulose ethers, the hydroxyl groups of the cellulose are typically reacted with alkali such as sodium hydroxide, followed by one or more etherifying agents such as methyl chloride, ethyl chloride, ethylene oxide or propylene oxide. Cellulose esters are commonly derived from cellulose through the reaction of the hydroxyl groups with organic acids, anhydrides, or acid chlorides. The cellulose derivative may be provided in any form, including but not limited to: powder and granular forms.

Preferably, the composition of the nicotine oral delivery product of the present invention comprises a cellulose derivative. Preferably, the cellulose derivative is a cellulose ether or cellulose ester.

Suitable cellulose ethers include, but are not limited to the following: alkyl cellulose ethers such as methylcellulose, ethylcellulose and ethyl methyl cellulose; hydroxyalkyl cellulose ethers such as hydroxyethyl cellulose, hydroxypropyl cellulose, hydroxyethyl methyl cellulose, hydroxypropyl methyl cellulose and ethyl hydroxyethyl cellulose; and carboxymethyl cellulose ethers such as carboxymethylcellulose.

Suitable cellulose esters include, but are not limited to the following: organic cellulose esters such as cellulose acetate, cellulose triacetate, cellulose propionate, cellulose acetate-propionate and cellulose acetate-butyrate; and inorganic cellulose esters such as nitrocellulose (cellulose nitrate) and cellulose sulfate.

Preferably, the cellulose derivative of the composition of the nicotine oral delivery product of the present invention is a cellulose ether. More preferably, the cellulose derivative is carboxymethylcellulose. It is surprisingly noted by the applicant that the use of carboxymethylcellulose as the cellulose or cellulose derivative further advantageously provides the nicotine oral delivery product of the present invention with a neutral taste, which is welcomed by the user. By “neutral taste” is meant a taste that is pleasant to the user, and allows any selected one or more flavour introduced into the composition of the nicotine oral delivery product to be the predominant taste associated with the nicotine oral delivery product. It will be appreciated by a skilled person that carboxymethylcellulose may be used in its sodium salt form, i.e. as sodium carboxymethylcellulose. Carboxymethyl cellulose is commercially available under the WALOCEL™ range, such as WALOCEL™ CRT 100 GA from DuPont. When the cellulose derivative is carboxymethylcellulose, the carboxymethylcellulose is preferably in its sodium salt form, sodium carboxymethylcellulose. Preferably, the cellulose derivative is sodium carboxymethylcellulose.

It has been further surprisingly and advantageously found that effective delivery of nicotine to the user, optimal moisture content, pleasant taste and mouth-feel, and avoidance of discolouration of a user's teeth and gums demonstrated by the nicotine oral delivery product of the present invention may result from the presence of a cellulose or cellulose derivative in the composition of the nicotine oral delivery product in granular form. This is embodied by an aspect of the present invention, but is also applicable to all other aspects of the present invention.

The term “cellulose” as used herein encompasses compounds of cellulose having different molecular mass (degree of polymerisation) and crystallinity. The term further encompasses partially depolymerised compounds of cellulose and purified compounds of cellulose, for example, a purified partially depolymerised compound of a-cellulose precursor. The cellulose may be provided in any form, including but not limited to powder and granular forms.

The cellulose or cellulose derivative of the composition of the nicotine oral delivery product of the present invention may be in granular form. The cellulose or cellulose derivative of the composition of the nicotine oral delivery product of the present invention may be introduced into the composition in granular form. Preferably, the cellulose or cellulose derivative is in granular form. It is surprisingly noted by the applicant that the use of a cellulose or cellulose derivative in granular form further advantageously provides the nicotine oral delivery product of the present invention with a neutral taste, which is welcomed by the user. It is further noted by the applicant that the use of a cellulose or cellulose derivative in granular form further advantageously reduces dust produced during manufacture of the nicotine oral delivery product of the present invention.

By “granular form” and like terms used herein is meant that the component is present in the composition as granules. The component is introduced into the composition as granules. It will be appreciated by a skilled person that the component in granular form may be formed by a granulation process, such as wet or dry granulation, during which single discrete particles such as powder particles agglomerate to form larger entities, i.e. granules. The granular form is therefore to be physically distinguished from powder form. Typically, upon measurement of particle size distribution of a component in granular form by sieve analysis, the main distribution (at least 50 wt % of the granules) is from 0.2 to 1 mm, such as from 0.5 to 1 mm, or from 0.5 to 0.7 mm.

Preferably, the cellulose or cellulose derivative is a cellulose derivative in granular form. More preferably, the cellulose or cellulose derivative is a cellulose ether in granular form. More preferably, the cellulose or cellulose derivative is carboxymethylcellulose or its sodium salt, sodium carboxymethylcellulose, in granular form. Most preferably, the cellulose or cellulose derivative is sodium carboxymethylcellulose in granular form.

It has been surprisingly and advantageously found that when the cellulose or cellulose derivative is a cellulose derivative in granular form, such as a cellulose ether in granular form, or carboxymethylcellulose or its sodium salt, sodium carboxymethylcellulose, in granular form, the nicotine oral delivery product of the present invention demonstrates effective delivery of nicotine to the user, optimal moisture content, pleasant taste and mouth-feel, and avoidance of discolouration of a user's teeth and gums. In addition, the nicotine oral delivery product of the present invention advantageously has a neutral taste, which is welcomed by the user. By “neutral taste” is meant a taste that is pleasant to the user, and allows any selected one or more flavour introduced into the composition of the nicotine oral delivery product to be the predominant taste associated with the nicotine oral delivery product.

It is noted that the cellulose or cellulose derivative of the composition of the nicotine oral delivery product of the present invention functions as a filler for the composition of the nicotine oral delivery product of the present invention.

The cellulose or cellulose derivative may be present in the composition of the nicotine oral delivery product of the present invention in any suitable amount. Preferably, the composition of the nicotine oral delivery product comprises from 1 to 20 wt %, such as from 5 to 20 wt %, or even from 5 to 15 wt % of the cellulose or cellulose derivative.

The composition of the nicotine oral delivery product of the present invention may further comprise an additional cellulose or additional cellulose derivative.

The composition of the nicotine oral delivery product of the present invention may comprise additional cellulose. The term “additional cellulose” encompasses compounds of cellulose having different molecular mass (degree of polymerisation) and crystallinity. The term further encompasses partially depolymerised compounds of cellulose and purified compounds of cellulose, for example, a purified partially depolymerised compound of α-cellulose precursor. The additional cellulose may be provided in any form, including but not limited to powder and granular forms.

The composition of the nicotine oral delivery product of the present invention may comprise additional cellulose derivative. The additional cellulose derivative may be any suitable cellulose derivative. The term “additional cellulose derivative” as used herein in relation to the composition of the nicotine oral delivery product of the present invention, refers to cellulose which has been modified by virtue of the hydroxyl groups of the compound being partially or fully reacted with one or multiple reagents. Examples of suitable additional cellulose derivatives include cellulose ethers and cellulose esters. In the production of cellulose ethers, the hydroxyl groups of the cellulose are typically reacted with alkali such as sodium hydroxide, followed by one or more etherifying agents such as methyl chloride, ethyl chloride, ethylene oxide or propylene oxide. Cellulose esters are commonly derived from cellulose through the reaction of the hydroxyl groups with organic acids, anhydrides, or acid chlorides. The additional cellulose derivative may be provided in any form, including but not limited to powder and granular forms.

The additional cellulose or additional cellulose derivative may be in powder form. By “powder form” and like terms used herein is meant that the component is present in the composition as a powder. The component is introduced into the composition as a powder. Powder refers to particulate material having a relatively small particle size. Typically, for powder, upon measurement of particle size distribution of the component by sieve analysis, the main distribution (at least 50 wt % of the particles) is from 0.01 to 0.5 mm, such as from 0.01 to 0.2 mm, or from 0.01 to 0.1 mm. It will be appreciated by a skilled person that a component in powder form has not been subjected to a granulation process. The powder form of a component is therefore to be physically distinguished from granular form.

Examples of suitable additional cellulose derivatives include cellulose ethers and cellulose esters.

Suitable cellulose ethers include, but are not limited to the following: alkyl cellulose ethers such as methylcellulose, ethylcellulose and ethyl methyl cellulose; hydroxyalkyl cellulose ethers such as hydroxyethyl cellulose, hydroxypropyl cellulose, hydroxyethyl methyl cellulose, hydroxypropyl methyl cellulose and ethyl hydroxyethyl cellulose; and carboxymethyl cellulose ethers such as carboxymethylcellulose. It will be appreciated by a skilled person that carboxymethylcellulose may be used in its sodium salt form, i.e. sodium carboxymethylcellulose. Preferably, carboxymethylcellulose is utilised in its sodium salt form, sodium carboxymethylcellulose.

Suitable cellulose esters include, but are not limited to the following: organic cellulose esters such as cellulose acetate, cellulose triacetate, cellulose propionate, cellulose acetate-propionate and cellulose acetate-butyrate; and inorganic cellulose esters such as nitrocellulose (cellulose nitrate) and cellulose sulfate.

Preferably, the additional cellulose or additional cellulose derivative is cellulose. More preferably, the additional cellulose or additional cellulose derivative is microcrystalline cellulose. It is surprisingly noted by the applicant that the use of microcrystalline cellulose as the additional cellulose or additional cellulose derivative further advantageously provides the nicotine oral delivery product of the present invention with a neutral taste, which is welcomed by the user.

It is noted that the additional cellulose or additional cellulose derivative of the composition of the nicotine oral delivery product of the present invention functions as a filler for the composition of the nicotine oral delivery product of the present invention.

Preferably, the composition of the nicotine oral delivery product of the present invention comprises the additional cellulose or additional cellulose derivative.

Preferably, the additional cellulose or additional cellulose derivative is cellulose in powder form. More preferably, the additional cellulose or additional cellulose derivative is microcrystalline cellulose in powder form.

It has been surprisingly and advantageously found that when the composition of the nicotine oral delivery product of the present invention comprises the additional cellulose or additional cellulose derivative, and the additional cellulose or additional cellulose derivative is cellulose in powder form, the nicotine oral delivery product of the present invention demonstrates effective delivery of nicotine to the user, optimal moisture content, pleasant taste and mouth-feel, and avoidance of discolouration of a user's teeth and gums. In addition, the nicotine oral delivery product of the present invention advantageously has a neutral taste, which is welcomed by the user. By “neutral taste” is meant a taste that is pleasant to the user, and allows any selected one or more flavour introduced into the composition of the nicotine oral delivery product to be the predominant taste associated with the nicotine oral delivery product.

Preferably, the composition of the nicotine oral delivery product of the present invention comprises a cellulose ether as the cellulose or cellulose derivative and cellulose as the additional cellulose or additional cellulose derivative. Preferably, the cellulose ether is in granular form. Preferably, the cellulose is in powder form.

Preferably, the composition of the nicotine oral delivery product of the present invention comprises carboxymethylcellulose, or sodium carboxymethylcellulose, as the cellulose or cellulose derivative and microcrystalline cellulose as the additional cellulose or additional cellulose derivative. Preferably, the carboxymethylcellulose, or sodium carboxymethylcellulose, is in granular form. Preferably, the microcrystalline cellulose is in powder form.

The additional cellulose or additional cellulose derivative may be present in the composition of the nicotine oral delivery product of the present invention in any suitable amount. Preferably, the composition comprises from 1 to 30 wt %, such as from 10 to 30 wt %, or even from 12 to 30 wt % of the additional cellulose or additional cellulose derivative.

The composition of the nicotine oral delivery product of the present invention may further comprise one or more pH-adjusting agents. The term “pH adjusting agent” and like terms used herein refers to agents which adjust and regulate the pH of the composition to which they are added. Such agents may be acids and bases, including acidic and alkaline buffering agents. However, the term does not encompass substances that only affect pH by dilution.

Any pharmaceutically acceptable pH adjusting agent may be utilised in the composition of the nicotine oral delivery product of the present invention. Suitable examples of pH adjusting agents include, but are not limited to: carbonates including monocarbonates, bicarbonates and sesquicarbonates of alkaline metals or ammonium such as sodium carbonate, potassium carbonate, potassium bicarbonate, sodium bicarbonate and magnesium carbonate; acetates of alkaline metals or ammonium; glycinates of alkaline metals or ammonium; gluconates of alkaline metals or ammonium, borates of alkaline metals or ammonium, glycerophosphates of alkaline metals or ammonium; citrates of alkaline metals or ammonium; phosphate systems including monohydrogenphosphate, dihydrogenphosphate and trihydrogenphosphate; metal hydroxides such as sodium hydroxide and potassium hydroxide; and mixtures thereof. Preferably, the composition of the nicotine oral delivery product of the present invention comprises one or more pH-adjusting agents. Preferably, the one or more pH-adjusting agent is selected from sodium carbonate, potassium carbonate and potassium bicarbonate. More preferably, the one or more pH-adjusting agent is sodium carbonate. It is surprisingly noted by the applicant that the use of sodium carbonate as the one or more pH-adjusting agent further advantageously enables the neutral taste of the nicotine oral delivery product of the present invention with a neutral taste, which is welcomed by the user.

It will be appreciated by a skilled person that the absorption of nicotine over the mucous membrane in the oral cavity of a user is effected by local pH, i.e. the pH inside and in close proximity to the nicotine oral delivery product in the saliva of the user. Preferably, the one or more pH-adjusting agent provides a local pH of 6 or above, such as a pH of from 8 to 10, more preferably 9, when the composition of the nicotine oral delivery product is dissolved or dispersed in aqueous solution such as purified water.

It will be appreciated by a person skilled in the art that the amount of pH-adjusting agent present in the composition affects local pH. The one or more pH-adjusting agent may be present in the composition of the nicotine oral delivery product of the present invention in any suitable amount. Preferably, the composition comprises from 1 to 10 wt %, such as from 2 to 8 wt % of the one or more pH-adjusting agent.

The composition of the nicotine oral delivery product of the present invention may further comprise one or more additional fillers. Suitable examples of additional fillers include but are not limited to: polysaccharides such as maltitol, polyols, natural fibres, and mixtures thereof. It is noted that an additional filler may also have a secondary function as, for example, a sweetener. Preferably, the composition of the nicotine oral delivery product of the present invention is free of sugars, in light of the negative impact that they may have on a user's teeth. Preferably, the one or more additional filler is maltitol.

The one or more additional filler may be present in the composition of the nicotine oral delivery product of the present invention in any suitable amount. Preferably, the composition comprises from 20 to 60 wt %, such as from 30 to 50 wt % of the one or more additional filler.

The composition of the nicotine oral delivery product of the present invention may further comprise one or more flavour. By “flavour” and like terms used herein is meant a substance used to influence aroma and/or taste of the composition of the nicotine oral delivery product, including but not limited to, essential oils, single flavour compounds, compounded flavourings and extracts. Examples of suitable flavours include bergamot, eucalyptus, orange, mandarin, citrus, lemon, peppermint, spearmint, mint, menthol, liquorice, wintergreen, whiskey, rum, cherry, various berries, tobacco, coffee, vanilla, lime, apple, peach, carvone, limonene and any combination thereof.

The one or more flavour may be present in the composition of the nicotine oral delivery product of the present invention in any suitable amount. Preferably, the composition comprises from 1 to 6 wt %, such as from 1 to 4 wt % of the one or more flavour.

The composition of the nicotine oral delivery product may further comprise one or more humectants. Humectants may also be known in the art as moisturisers or softeners and are present in the composition of the nicotine oral delivery product of the present invention to control and maintain appropriate moisture levels of the composition. Suitable examples of humectants include, but are not limited to: glycerol, glycerine, propylene glycol, hexyleneglycol, butylene glycol, polydextrose, sorbitol, maltitol, xylitol, glyceryl triacetate, triethylene glycol and combinations thereof. Preferably, the one or more humectant is selected to be glycerol, propylene glycol, or a combination thereof. It will be appreciated by a skilled person that the amount of propylene glycol in the composition will be in accordance with associated safety regulations. More preferably, the one or more humectant is glycerol.

The one or more humectants may be present in the composition of the nicotine oral delivery product of the present invention in any suitable amount. Preferably, the composition comprises from 1 to 25 wt %, such as from 1 to 15 wt %, such as from 5 to 15 wt % of the one or more humectant.

The composition of the nicotine oral delivery product of the present invention may further comprise one or more sweetener to enhance the taste and sweetness of flavour provided by the nicotine oral delivery product. Suitable examples of sweetener include, but are not limited to: polyols such as xylitol; mono-, di- and tri-polysaccharides such as mannitol and maltitol; natural and synthetic sweeteners such as sucrose, glucose, dextrose, maltose, fructose, saccharin, aspartame, acesulfame including acesulfame K, sucralose, saccharin and cyclamtes and mixtures thereof.

The one or more sweetener may be present in the composition of the nicotine oral delivery product of the present invention in any suitable amount. The composition may comprise from 1 to 20 wt % of the one or more sweetener.

The composition of the nicotine oral delivery product of the present invention is enclosed in a water insoluble pouch, i.e. completely contained within a water insoluble pouch. The composition of the nicotine oral delivery product is sealed within a water insoluble pouch. The water insoluble pouch may be formed from any suitable material. The water insoluble pouch may be formed from any suitable pharmaceutically acceptable material. The term “pharmaceutically acceptable” as used herein refers to non-toxic compounds or materials that are biocompatible and physiologically acceptable, and do not decrease the effectiveness of the biological activity of the active ingredient, i.e. nicotine. Such pharmaceutically acceptable materials for the water insoluble pouch will be well known in the art. Examples of suitable materials from which the water insoluble pouch may be formed include but are not limited to: woven or non-woven fabrics such as cotton or fleece; and heat-sealable non-woven cellulose or other polymeric materials such as synthetic, semi-synthetic or natural polymeric agents. The water insoluble pouch may further comprise components, such as polymers, to bind the materials and facilitate sealing of the pouch. Preferably, the water insoluble pouch of the nicotine oral delivery product of the present invention is formed from a non-woven material comprising viscose rayon fibres (i.e. regenerated cellulose) and an acrylic polymer that acts as a binder for the non-woven material and provides for heat-sealing of the pouch during manufacturing thereof.

The water insoluble pouch is sealed to enclose and retain the composition with the pouch. Preferably, the water insoluble pouch is heat-treated to seal the composition inside and form the nicotine oral delivery product of the present invention.

It will be appreciated that the material of the water insoluble pouch influences the mouth-feel of the nicotine oral delivery product for the user. It will further be appreciated that the water insoluble pouch: does not dissolve in saliva; is chemically and physically stable; is pharmaceutically acceptable; is insoluble in water i.e. incapable of being dissolved in water; is easily fillable with the composition; and is easily sealable. The water insoluble pouch of the nicotine oral delivery product provides a semi-permeable membrane which prevents the composition leaving the pouch, but when the product is in use and placed in a user's mouth, is permeable to saliva and dissolved components of the composition including nicotine and flavour components such that they can diffuse therefrom into the user's mouth.

It will be appreciated that the nicotine oral delivery product of the present invention is intended to be placed in the oral cavity of a user, such as by buccal placement (placing the product between the upper or lower gum and the lip or cheek). The nicotine oral delivery product will therefore be sized and configured to fit comfortably and discreetly in the user's mouth in this manner. It may have an oblong shape, such as a rectangular shape.

The composition may be enclosed in the water insoluble pouch of the nicotine oral delivery product of the present invention in any suitable amount. Preferably 50 to 1,000 mg, such as 200 to 800 mg of the composition is enclosed in the water insoluble pouch. It will be appreciated that the amount of the composition enclosed in the water insoluble pouch is selected such that the nicotine oral delivery product remains of an appropriate size.

The total weight of the nicotine oral delivery product (both composition and water insoluble pouch) according to the present invention may be from 0.3 to 1.5 g, such as from 0.3 to 1 g, or even from 0.4 to 0.8 g.

It has been surprisingly and advantageously found by the applicant that the nicotine oral delivery product of the present invention effectively delivers nicotine to a user. The nicotine oral delivery product of the present invention provides a high perceived nicotine impact. The term “perceived nicotine impact” as used herein refers to the nicotine ‘hit’ perceived by a user. As discussed above, the local pH of 6 or above, such as from 8 to 10, or even 9, contributes to the absorption of nicotine over the mucous membrane in the oral cavity of a user.

Further, the applicant has surprisingly and advantageously found that the composition of the nicotine oral delivery product of the present invention has a moisture content of from 7 to 12 wt %, preferably 9 wt %. Such a moisture content is optimal, enabling the nicotine oral delivery product of the present invention to have an extended shelf life such that the composition does not dry out, whilst also preventing the composition of the nicotine oral delivery product from being too dry such that excessive dust is formed during the manufacture thereof. By the term “moisture content” as used herein is meant the total amount of oven volatile materials in the composition. It will be appreciated by a skilled person that in the context of the present invention, moisture content is provided by the one or more humectants and flavours, predominantly the one or more humectants. Compositions for nicotine oral delivery products known in the art tend to have a much higher moisture content than that of the products of the present invention. In the context of the present invention, the moisture content of the composition of the nicotine oral delivery product is measured using a Mettler Toledo's Moisture Analyzer HB43, a balance with halogen heating technology. The sample is heated to 105° C. The measurement is stopped when the weight change is less than 1 mg during a 90 seconds period. The moisture content as a weight percentage of the sample is then calculated automatically by the Moisture Analyzer HB43.

Still further, it will be appreciated by a skilled person that the composition of the nicotine oral delivery product of the present invention does not contain water. Accordingly, microorganisms are prevented from growing, providing a longer shelf life for the nicotine oral delivery products of the present invention.

The nicotine oral delivery product of the present invention further has a pleasant taste to the user. As discussed above, the components of the composition, in particular the cellulose or cellulose derivative and additional cellulose or additional cellulose derivative, contribute to the neutral taste of the nicotine oral delivery product, which is welcomed by a user, with any additional flavours or sweeteners in the composition of the nicotine oral delivery product of the present invention providing the predominant enjoyable taste to the user.

During use, the nicotine oral delivery product is placed and retained for a period of time in the mouth of a user. This is commonly between the upper or lower gum of the user and the chin or cheek. Once saliva enters the interior of the pouch, it dissolves any components of the composition enclosed therein that are soluble in saliva. These solubilised components of the composition (including nicotine, humectants and flavour components) diffuse from the pouch in the saliva and into the mouth of the user. Once released from the pouch, the nicotine is bioavailable, i.e. available for absorption over a mucous membrane in the oral cavity of the user. Accordingly, once released from the pouch, nicotine is absorbed over a mucous membrane of the oral cavity of a user into their blood so as to provide nicotine and satisfaction to the user.

It is noted that the nicotine oral delivery products of the present invention are intended for single use.

As used herein, the terms “mouth” and “oral cavity” are interchangeable.

According to a further aspect of the present invention, there is provided a method of making a nicotine oral delivery product containing a composition enclosed in a water insoluble pouch, wherein said pouch is permeable for saliva and dissolved components of the composition, the composition comprising a source of nicotine and a cellulose derivative, and wherein the method comprises enclosing the composition in a water insoluble pouch.

According to a further aspect of the present invention, there is provided a method of making a nicotine oral delivery product containing a composition enclosed in a water insoluble pouch, wherein said pouch is permeable for saliva and dissolved components of the composition, the composition comprising a source of nicotine and a cellulose or cellulose derivative, wherein the cellulose or cellulose derivative is in granular form, and wherein the method comprises introducing the cellulose or cellulose derivative into the composition in granular form, and enclosing the composition in a water insoluble pouch.

For these further aspects of the present invention, the nicotine oral delivery product, composition and water insoluble pouch may have any of the features described herein with regard to all other aspects of the present invention.

The method of making the nicotine oral delivery product according to these further aspects of the present invention may comprise the steps of admixing the components of the composition, filling the composition into the water insoluble pouch, and enclosing the composition in the water insoluble pouch such that the composition is completely contained within. Preferably, the water insoluble pouch is sealed by heat to enclose and retain the composition therein. Appropriate machines for the manufacture of the nicotine oral delivery products of the present invention will be well known in the art.

By “introducing the cellulose or cellulose derivative into the composition in granular form” and like terms and phrases used herein, is meant that the cellulose or cellulose derivative is in granular form when it is added to the mixture of other components that form the composition.

It will be appreciated by a skilled person that, in some arrangements, during manufacture of the nicotine oral delivery product, the size, shape and other characteristics such as malleability and hardness of the composition and particular solid components thereof, such as the cellulose or cellulose derivative, may be modified upon admixing of the components of the composition.

According to a still further aspect of the present invention, there is provided a nicotine oral delivery product containing a composition enclosed in a water insoluble pouch, wherein said pouch is permeable for saliva and dissolved components of the composition, the composition comprising a source of nicotine and a cellulose or cellulose derivative, wherein the cellulose or cellulose derivative is introduced into the composition in granular form.

For this still further aspect of the present invention, the nicotine oral delivery product, composition and water insoluble pouch may have any of the features described herein with regard to all other aspects of the present invention.

All of the features contained herein may be combined with any of the above aspects and in any combination.

All references to chemical compounds herein are to be interpreted as covering the compounds per se, and also, where appropriate, derivatives, hydrates, solvates, complexes, isomers and tautomers thereof.

All references to “wt %” as used herein refer to the percentage by weight relative to the total weight of the composition enclosed within the water insoluble pouch of the nicotine oral delivery product of the present invention.

For a better understanding of the present invention, and to show how embodiments of the same may be carried into effect, reference will now be made, by way of example, to the following experimental data.

### EXAMPLES

A composition was prepared by admixing the components listed in Table 1.

TABLE 1

| Component | Wt % |
|---|---|
| Nicotine polacrilex 20% (source of nicotine) | 7.40 |
| CRT 100 GA (sodium carboxymethylcellulose, a | 10.44 |
| cellulose derivative in granular form) | |
| Maltitol (filler) | 41.81 |
| Microcrystalline cellulose (additional cellulose in | 20.49 |
| powder form) | |
| Sodium carbonate (pH adjusting agent) | 5.86 |
| Mint flavouring (flavour) | 3.30 |
| Glycerol (humectant) | 10.70 |

615 mg of the composition was introduced into a water insoluble pouch formed of viscose rayon fibres bound by an acrylic binder, and enclosed and retained therein by heat sealing of the water insoluble pouch to form a nicotine oral delivery product according to the present invention. The total weight of the nicotine oral delivery product was 0.65 g.

The nicotine oral delivery product provided a high perceived nicotine impact when tested by 15 volunteers. Further, the nicotine oral delivery product was found to have a pleasant taste and mouth-feel. A local pH of 9 and moisture content of 9 wt % was also measured, demonstrating the advantageous properties of the nicotine oral delivery products of the present invention.

1. A nicotine oral delivery product comprising a composition enclosed in a water insoluble pouch, wherein said pouch is permeable for saliva and dissolved compounds of the composition, the composition comprising a source of nicotine and a cellulose or cellulose derivative, wherein the cellulose or cellulose derivative is in granular form.

2. The nicotine oral delivery product according to claim 1, wherein the cellulose or cellulose derivative is a cellulose derivative.

3. The nicotine oral delivery product according to claim 2, wherein the cellulose derivative is a cellulose ether.

4. The nicotine oral delivery product according to claim 2, wherein the cellulose derivative is carboxymethylcellulose.

5. The nicotine oral delivery product according to claim 1, wherein the source of nicotine provides 0.1 to 30 mg of nicotine per nicotine oral delivery product.

6. The nicotine oral delivery product according to claim 5, wherein the source of nicotine is at least one selected from the group of tobacco, nicotine salt, nicotine base, stabilised nicotine and combinations thereof.

7. The nicotine oral delivery product according to claim 1, wherein the source of nicotine is at least one selected from the group of nicotine salt, nicotine base, stabilised nicotine and combinations thereof.

8. The nicotine oral delivery product according to claim 1, wherein the source of nicotine is stabilised nicotine.

9. The nicotine oral delivery product according to claim 8, wherein the source of nicotine is nicotine polacrilex.

10. The nicotine oral delivery product according to claim 1, wherein the composition further comprises an additional cellulose or additional cellulose derivative.

11. The nicotine oral delivery product according to claim 10, wherein the additional cellulose or additional cellulose derivative is cellulose.

12. The nicotine oral delivery product according to claim 10, wherein the additional cellulose or additional cellulose derivative is microcrystalline cellulose.

13. The nicotine oral delivery product according to claim 10, wherein the additional cellulose or additional cellulose derivative is in powder form.

14. A method of making the nicotine oral delivery product according to claim 1, the method comprising introducing the cellulose or cellulose derivative into the composition in granular form, and enclosing the composition in the water insoluble pouch.

15. (canceled)

16. The nicotine oral delivery product according to claim 2, wherein the cellulose derivative is sodium carboxymethylcellulose.
